(19) United States
(12) Patent Application Publication
(10) Pub. No.: US 2008/0226616 A1
(43) Pub. Date: Sep. 18, 2008

(54) Emulsifier Combination for Cosmetics

(76) Inventors: Jens Schulz, Schenefeld (DE); Jens Nielsen, Henstedt-Ulzburg (DE); Rainer Kroepke, Schenefeld (DE); Gunhild Hamer, Hamburg (DE); Astrid Heptner, Hamburg (DE); Svea Behrens, Hamburg (DE)

Correspondence Address: GREENBLUM & BERNSTEIN, P.L.C., 1950 ROLAND CLARKE PLACE, RESTON, VA, 20191, US

(21) Appl. No.: 11/547,104

(22) PCT Filed: Jul. 7, 2006
(86) PCT No.: PCT/EP2006/064003

§ 371 Date: Dec. 11, 2006

(30) Foreign Application Priority Data
Jul. 7, 2005 (DE) 10 2005 032237.9

(51) Int. Cl. A61K 38/43; A61K 31/205; A61K 31/197; A61K 31/194; A61K 47/32; A61K 31/437; A61K 31/519; A61P 17/16; A61P 17/18; A61P 17/02

(52) U.S. Cl. 424/94.1; 514/574; 514/772.6; 514/556; 514/565; 514/299; 514/252.16

(57) ABSTRACT

The present invention relates to a cosmetic preparation containing an emulsifier combination of polyacrylic acid salts, acrylate/C10-30 alkyl acrylate crosspolymer and carrageenan and the use of the cosmetic preparation.

The present invention relates to a cosmetic preparation containing an emulsifier combination of polyacrylic acid salts, acrylate/C10-30 alkyl acrylate crosspolymer and carrageenan and the use of the cosmetic preparation.

The desire to look beautiful and attractive is naturally deeply rooted in human nature. Even though the beauty ideal has seen some changes over time, striving for a flawless exterior has always been a human goal. The condition and the appearance of the skin plays an important part in a beautiful and attractive appearance.

In order for the skin to completely fulfill its biological functions, it requires regular cleansing and care. Cleansing the skin thereby serves to remove dirt, perspiration and residue of dead skin particles which form an ideal breeding ground for pathogens and parasites of all types. Skincare products generally serve to moisturize and regrease the skin. Active substances are often added to them which regenerate the skin and, for example, are intended to prevent and reduce its premature ageing (e.g., the appearance of wrinkles, lines).

The skin needs water-soluble treatment agents. Skin treatments therefore nowadays generally contain an oil/adipoid and an aqueous phase.

By nature, fats and oils are immiscible with water and water-soluble substances (e.g., salts). In order to obtain an optically homogeneous stable preparation from water and oil phase, surface-active agents (emulsifiers) have to be added to them which are able to distribute the smallest oil and solid globules in water (oil in water emulsion, O/W emulsion) or water droplets in oils (water in oil emulsion, W/O emulsion). The emulsions produced permit a “2 in 1” care of the skin with water-soluble and oil-soluble substances. Depending on the consistency, they are described as ointments or creams (viscous to cutting consistency) or lotions (low viscosity).

Emulsions have excellent cosmetic properties: the lipophilic constituents are absorbed into the skin relatively quickly, the aqueous constituents increase the skin moisture in a long-lasting manner.

The properties of a cosmetic emulsion depend chiefly on the emulsifiers used. The oldest known emulsifiers include soaps, such as, e.g., sodium stearate.

Emulsifiers and emulsifier systems from the prior art have a number of drawbacks:
The emulsifiers have to emulsify preparation constituents of various compositions and with different properties such as polarity (e.g., with oils) or ionic strength (with saline constituents of the aqueous phase) into an optically homogeneous emulsion quickly and in a stable manner over a long period. In order to meet these requirements, the emulsifier system often has to be individually matched to the composition of the emulsion.
The emulsifiers should be well tolerated by the skin and should not irritate the skin.
The emulsifiers should not penetrate into the skin.

It was therefore the object of the present invention to eliminate the defects of the prior art and to develop an improved emulsifier system that meets these requirements.

Conventional emulsifiers are composed of an aqueous phase and a lipid phase. There is a great demand for preparations that are particularly “light” and low in fat and contain at most small amounts of lipids. However, according to the prior art no lipophilic active substances (or at most, insufficient amounts thereof) can be incorporated into such preparations.

It was therefore the object of the present invention to develop a “low-fat” (i.e., largely free from lipids of all kinds) cosmetic preparation, in which larger amounts of lipophilic active substances can be incorporated.

Furthermore, the object is to not unbalance the natural lipometabolism of the skin through the application of cosmetics.

It was therefore the object of the present invention to develop a cosmetic preparation in which larger amounts of lipophilic active substances are incorporated, but which do not unbalance the natural lipometabolism of the skin.

Surprisingly, the objects are attained through a cosmetic preparation comprising:
a) 0.01 to 1.5% by weight of polyacrylic acid salts,
b) 0.01 to 1.5% by weight of cross-linked polyacrylic acid salts,
c) 0.01 to 1.5% by weight of acrylate/C10-30 alkyl acrylate crosspolymer,
d) 0.01 to 2.5% by weight of carrageenan,
   the weights given relating to the total weight of the cosmetic preparation.

The preferred polyacrylic acid salt according to the invention is sodium polyacrylate (CAS: 9003-04-7) which is available, e.g., under the trade name COSMEDIA SP from Cognis. These polyacrylic acid salts are not cross-linked.

It is advantageous according to the invention to use carbomers as cross-linked polyacrylic acid salts. The preferred cross-linked polyacrylic acid salt is sodium carbomer (e.g., CAS: 9007-16-3, CAS: 9007-17-4, CAS: 9062-04-8, CAS: 7062-04-8, CAS: 76050-42-5), which is available, e.g., under the trade name Carbopol 980 from Noveon.

The acrylate/C10-30 alkyl acrylate crosspolymer (INCI: acrylates/C10-30 alkyl acrylates crosspolymer) preferred according to the invention is available, e.g., under the trade name Permulen TR-1 from Noveon.

The preferred carrageenan according to the invention is registered under CAS no. 9000-07-1 (EINECS no. 232-524-2) and is available, e.g., under the trade name Gelcarin GP-379NF from Interorgana.

Embodiments of the cosmetic preparation according to the invention which are preferred according to the invention are characterized in that they comprise as further component 0.01 to 2.5% by weight of vinylpyrrolidone/vinyl acetate copolymer (VP/VA copolymer), based on the total weight of the cosmetic preparation.

The vinylpyrrolidone/vinyl acetate copolymer (VP/VA copolymer) that is preferred according to the invention is a polymer which is formed of 60% of vinylpyrrolidone and 40% of vinyl acetate and is available, e.g., under the trade name Luviskol VA64 W from BASF.

The preparation according to the invention is advantageously characterized according to the invention in that the weight ratio of polyacrylic acid salts to acrylates/C10-30 alkyl acrylates crosspolymer is from 1:10 to 10:1 and preferably from 1:1 to 2:1.

The preparation according to the invention is advantageously characterized according to the invention in that the weight ratio of polyacrylic acid salts to carrageenan is from 1:10 to 10:1.

The preparation according to the invention is advantageously characterized according to the invention in that the weight ratio of polyacrylic acid salts to vinyl pyrrolidone/vinyl acetate copolymers is from 1:10 to 10:1 and preferably from 1:1 to 2:1.

A cosmetic preparation that contains the emulsifier combination according to the invention of polyacrylic acid salts, cross-linked polyacrylic acid salts, acrylate/C10-30 alkyl acrylate crosspolymer and carrageenan in a total concentration of from 0.05 to 9.5% by weight, and preferably in a concentration of from 0.5 to 1.8% by weight, respectively based on the total weight of the cosmetic preparation, is also according to the invention.

The cosmetic preparation according to the invention is preferably an aqueous or aqueous-alcoholic gel or a gel cream.

Gel creams are particularly light products with a low emulsifier and lipid content. They are characterized in that the can be easily dispersed over the skin and provide a feeling of freshness. After the product has been applied, no residue or only a little residue should remain on the skin. Gel creams generally contain a relatively high proportion of hydrophilic thickening agents. Since the thickener or the thickener system is in the outer phase, it has a significant impact on the sensory properties of the product. Conventional thickener systems either cannot be dispersed, do not provide a feeling of freshness or leave behind an overly sticky residue on the skin.

The cosmetic preparation according to the invention is advantageously characterized according to the invention in that the preparation contains one or more active substances selected from the group of hydrophilic active substances, whereby one or more active substances from the group of compounds carnitine, ubiquinone Q10, alpha-glucosylrutin, creatine, creatinine, caffeine, flavonoids, folic acid and 8-hexadecene-1,16-dicarboxylic acid (dioic acid, CAS no. 20701-68-2; provisional INCI name octadecendioic acid) are preferably used according to the invention.

Cosmetic preparations containing active substances according to the invention of this type contain the active substances according to the invention advantageously in a total concentration of from 0.0001 to 12.5% by weight and preferably in a total concentration of from 0.01 to 0.8% by weight, respectively based on the total weight of the preparation.

The aqueous phase of the preparations according to the invention can advantageously contain conventional cosmetic auxiliaries, such as alcohols, in particular those having a low C number, preferably ethanol and/or isopropanol, diols or polyols having a low C number and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and/or active substances such as self-tanners, repellents, vitamins, plant extracts, niacinamide, panthenol.

According to the invention, the preparation according to the invention also contains as further constituents alpha hydroxy acids and/or salts thereof.

According to the invention, lactic acid/lactate or citric acid/citrate in a concentration of 0.01 to 5% by weight, based on the total weight of the preparation are preferably used.

The preparation according to the invention can contain advantageously according to the invention further cosmetic additives, e.g., the UV light filters approved under the Cosmetics Directive, and preservative agents or preservative auxiliaries. Active ingredients of this type can be contained in the preparation in an advantageous manner according to the invention in concentrations of from 0.01 to 30% by weight, based on the total weight of the preparation.

The cosmetic preparation according to the invention advantageously contains according to the invention at least one water-soluble dye.

The cosmetic preparation according to the invention advantageously has a transparent or translucent appearance. Advantageously according to the invention, it can be stored in a transparent packaging.

It is advantageous for the purposes of the present invention if the cosmetic preparation according to the invention is free from surfactants with an HLB value greater than 25. It is preferred according to the invention if the cosmetic preparation according to the invention is free from surfactants with an HLB value greater than 20. The HLB values of surfactants can be taken from the usual tabular compilations (e.g., Fiedler, Lexikon der Hilfsstoffe für Pharmazie, Kosmetik und angrenzende Gebiete, Editio Cantor Verlag, Aulendorf).

An emulsion of a cosmetic preparation (preferably of an aqueous or aqueous-alcoholic gel) and the skin's own lipids is also according to the invention. An emulsion of this type is produced in that the cosmetic preparation (preferably of an aqueous and aqueous-alcoholic gel or gel cream) is applied to the skin and subsequently rubbed in.

The method for producing an emulsion of this type according to the invention which is characterized in that a cosmetic preparation according to the invention (preferably of an aqueous or aqueous-alcoholic gel or gel cream) is applied to the skin and rubbed in, is according to the invention. The rubbing of the cosmetic preparation on the skin is thereby preferably carried out with the hands or fingers, but can also be carried out according to the invention with the aid of an applicator (e.g., a sponge or a massage head).

The preparation can be present advantageously according to the invention in the form of a low-viscosity, sprayable aqueous or aqueous-alcoholic solution, in the form of a gel, as an ointment, cream or lotion (optionally sprayable).

The preparation can also be used advantageously according to the invention as a spray or impregnation medium for a bandage or wipe. Bandages and wipes impregnated with the preparation according to the invention are therefore according to the invention.

The use of a cosmetic preparation according to the invention or of an emulsion made therefrom for the cosmetic prophylaxis or cosmetic treatment of skin irregularities and/or cellulite is according to the invention.

The use of a cosmetic preparation to produce an emulsion with the skin's own lipids for cosmetic prophylaxis or cosmetic treatment of skin irregularities and/or cellulite is according to the invention.

Within the scope of this disclosure, prophylaxis and treatment are understood to be exclusively cosmetic prophylaxis and treatment and on no account a therapeutic prophylaxis and treatment as defined by patent law.

The cosmetic prophylaxis or cosmetic treatment of cellulite according to the invention is advantageously carried out according to the invention in combination with massage devices, ultrasound, infrared light, textiles, bandages and/or occlusive films.

Not least the use of the cosmetic preparation according to the invention as a sauna product is according to the invention.

The following examples are intended to clarify the following invention without limiting it. The data always refer to % by weight, unless stated otherwise.

Gels

| | 1 | 2 | 3 | 4 | 5 |
|---|---|---|---|---|---|
| Polyacrylic acid, Na salt | 1.0 | 0.2 | 0.5 | 0.1 | 1.5 |
| (carbomer | | | | | |
| PEG 40 stearate | 0.5 | — | 0.75 | — | — |
| Carrageenan | 1.25 | 0.2 | 0.2 | 0.1 | 2.5 |
| Sodium polyacrylate | 0.2 | 0.2 | 0.3 | 0.5 | 1.5 |
| Vinylpyrrolidone/vinyl | 1.0 | — | 0.2 | — | — |
| acetate copolymers | | | | | |
| Acrylates/C10-30 alkyl | 1.5 | 0.2 | 0.15 | 1.5 | 0.75 |
| acrylate Crosspolymer | | | | | |
| Water-soluble pigment | 0.01 | 0.4 | — | — | — |
| Cyclomethicone | 1 | 8.5 | 5 | 0.5 | 3 |
| Dimethiconol | 0.5 | 0.5 | 0.75 | 1.25 | 1.0 |
| Cetylstearyl alcohol | — | — | 1 | 1 | — |
| Hydrogenated | 0.5 | — | 1.0 | — | 0.25 |
| polyisobutene | | | | | |
| Carnitine | 0.5 | 0.1 | 0.75 | 3.0 | 0.25 |
| Panthenol | 0.5 | — | 0.75 | 0.25 | 0.1 |
| Glyerol | 3.0 | 8.6 | 12.5 | 17.2 | 5.0 |
| Perfume | q.s. | q.s. | q.s. | q.s. | q.s. |
| Methylpropanediol | 4.5 | 2.0 | 0.5 | 1.5 | — |
| Propyl paraben | 0.3 | — | 0.25 | 0.15 | — |
| Ethanol | — | 10.0 | 5.0 | — | 3.5 |
| Water | ad 100 | ad 100 | ad 100 | ad 100 | ad 100 |

1.-12. (canceled)

13. A cosmetic composition comprising, based on a total weight of the composition,
(a) from 0.01% to 1.5% by weight of one or more salts of polyacrylic acid;
(b) from 0.01% to 1.5% by weight of one or more salts of crosslinked polyacrylic acid;
(c) from 0.01% to 1.5% by weight of acrylate/C10-30 alkyl acrylate crosspolymer; and
(d) from 0.01% to 2.5% by weight of carageenan.

14. The composition of claim 13, wherein the composition comprises (a) to (d) in a total concentration of at least 0.05% by weight.

15. The composition of claim 13, wherein the composition comprises (a) to (d) in a total concentration of at least 0.5% by weight.

16. The composition of claim 15, wherein the composition comprises (a) to (d) in a total concentration of not more than 1.8% by weight.

17. The composition of claim 15, wherein a weight ratio of [(a) plus (b)] to (c) is from 1:10 to 10:1.

18. The composition of claim 15, wherein a weight ratio of [(a) plus (b)] to (d) is from 1:10 to 10:1.

19. The composition of claim 13, wherein the composition further comprises from 0.01% to 2.5% by weight of a vinylpyrrolidone/vinyl acetate copolymer.

20. The composition of claim 13, wherein the composition further comprises one or more substances selected from carnitine, ubiquinone Q10, alpha-glucosylrutin, creatine, creatinine, caffeine, a flavonoid, folic acid and 8-hexadecene-1,16-dicarboxylic acid in a total concentration of from 0.0001% to 12.5% by weight, based on the total weight of the composition.

21. The composition of claim 20, wherein the composition comprises the one or more substances in a total concentration of from 0.1% to 0.8% by weight.

22. The composition of claim 20, wherein the composition comprises carnitine.

23. The composition of claim 22, wherein the composition comprises carnitine in a concentration of from 0.1% to 3.0% by weight.

24. The composition of claim 13, wherein the composition is free of surfactants having an HLB value of 25 or higher.

25. The composition of claim 13, wherein the composition further comprises water and a water-soluble dye.

26. The composition of claim 20, wherein the composition is substantially free of lipids.

27. An aqueous or aqueous-alcoholic gel which comprises the composition of claim 13.

28. The gel of claim 27, wherein the gel is transparent or translucent.

29. An aqueous or aqueous-alcoholic cosmetic gel, wherein the gel comprises carnitine and a total of from 0.5% to 1.8% by weight, based on a total weight of the composition, of
(a) from 0.01% to 1.5% by weight of one or more salts of polyacrylic acid;
(b) from 0.01% to 1.5% by weight of one or more salts of crosslinked polyacrylic acid;
(c) from 0.01% to 1.5% by weight of acrylate/C10-30 alkyl acrylate crosspolymer; and
(d) at least 0.01% by weight of carageenan.

30. The gel of claim 29, wherein the composition comprises carnitine in a concentration of from 0.1% to 3.0% by weight.

31. The gel of claim 29, wherein the composition is substantially free of lipids.

32. A cosmetic gel cream which comprises carnitine and a total of from 0.5% to 1.8% by weight, based on a total weight of the composition, of
(a) from 0.01% to 1.5% by weight of one or more salts of polyacrylic acid;
(b) from 0.01% to 1.5% by weight of one or more salts of crosslinked polyacrylic acid;
(c) from 0.01% to 1.5% by weight of acrylate/C10-30 alkyl acrylate crosspolymer; and
(d) at least 0.01% by weight of carageenan.

33. A method of reducing skin irregularities or cellulite, wherein the method comprises applying the composition of claim 13 to at least one of irregular skin and skin affected by cellulite.

34. A method of preventing skin irregularities or cellulite, wherein the method comprises applying to skin the composition of claim 13.
